(19) United States
(12) Patent Application Publication
(10) Pub. No.: US 2010/0280368 A1
(43) Pub. Date: Nov. 4, 2010

(54) Trocar tube, Trocar, Obturator and/or Rectoscope for the Transluminal Endoscopic Surgery Via Natural Body Orifices

(75) Inventors: Salman CAN, Fribourg (CH); Adam Fiolka, Iserlohn (DE); Armin Schneider, Munchen (DE); Hubertus Feussner, Munchen (DE)

Correspondence Address: WOMBLE CARLYLE SANDRIDGE & RICE, PLLC, ATTN: PATENT DOCKETING, P.O. BOX 7037, ATLANTA, GA, 30357-0037, US

(73) Assignee: Technische Universitat Munchen, Munchen (DE)

(21) Appl. No.: 12/714,087

(22) Filed: Feb. 26, 2010

Related U.S. Application Data
(63) Continuation-in-part of application No. PCT/EP2008/006936, filed on Aug. 22, 2008.

(30) Foreign Application Priority Data
Aug. 27, 2007 (DE) 10 2007 040 358.7

(51) Int. Cl. A61B 1/31; A61B 17/34; A61B 8/00; A61B 8/12

(52) U.S. Cl. 600/431; 604/170.03; 604/167.03; 600/104; 600/439

(57) ABSTRACT

A trocar tube, trocar, obturator, rectoscope is suitable for transluminal endoscopic surgery via natural body cavities. A trocar tube (2) has a distal section (22) for positioning at least in the region of or outside the body cavity, and a proximal section (21) for positioning within the human or animal, wherein at least the proximal section has a substantially rigid or inflexible curvature. An obturator (3) and a rectoscope (5) may be adapted for use with such a trocar tube for this purpose.

### RELATED APPLICATIONS

This is a Continuation-in-part of International Application PCT/EP2008/006936, filed 22-August 2008, which published as WO 2009/027065A1 on 5 Mar. 2009, and claims priority to DE 10 2007 040 358.7, filed 26 Aug. 2007. The contents of the aforementioned international and priority applications are incorporated by reference in their entirety.

### FIELD OF THE INVENTION

The present invention relates to a trocar tube, a trocar, an obturator and a rectoscope, in particular for the transluminal surgery via natural body orifices. A trocar tube according to the present invention and/or a respective trocar is provided with a distal portion to be positioned at least in the region of or outside the body orifice and a proximal portion to be positioned within the human or animal.

### BACKGROUND OF THE INVENTION

In endoscopic and laparoscopic surgery a point has been reached at which many procedures can be performed in a minimally invasive way. In future, it will be possible to perform surgical intervention in the peritoneal region probably without any incision of the abdominal wall. Access will then be gained via natural body orifices—i.e. transgastrically, transvaginally, transvesically or transcolonically. The first report on such a procedure (here: transgastric liver biopsy) is from the year 2004 [Kalloo A N, Singh V K, Jagannath S B, et al., Flexible transgastric peritoneoscopy: a novel approach to diagnostic and therapeutic interventions in the peritoneal cavity. Gastrointest Endosc 2004; 60; pp. 114-117]. An overview of studies carried out so far is listed in a recently published article [De la Fuente S G, De Maria E J, Reynolds J D, et al., New Developments in Surgery. Arch Surg 2007; 142; pp. 295-297]. Leading experts of the American Society for Gastrointestinal Endoscopy (ASGE) and of the Society of American Gastrointestinal and Endoscopic Surgeons (SAGES) agreed during a meeting in July 2005 that this procedure referred to as “Natural Orifice Transluminal Endoscopic Surgery (NOTES)” would provide significant improvements to the patient, such as less pain, faster convalescence and better cosmetics, compared to present laparoscopic methods.

In transluminal surgery, the instruments so far have been introduced into the abdominal cavity mostly via an artificially made orifice in the anterior gastric wall, i.e. transgastrically. Although the experimental use of NOTES is presently largely restricted to animal models, Rao et al. as the first group report on its use in humans during appendectomies [Rao G V, Reddy N, Transgastric appendectomy in humans. Oral presentation at the annual meeting of the Society of American Gastrointestinal and Endoscopic Surgeons (SAGES) 2006]. NOTES was used in the USA in 2007 for the first time for carrying out a transgastric and transvaginal cholecystectomy. In particular the retroflection of the endoscope/the surgical instruments necessary for the inspection of/intervention in the two upper quadrants of the abdomen resulting in an inversion of the endoscopic view, as well as complications relating to a safe closure of the gastric wall at the end of the intervention are mentioned as disadvantages of the transgastric access. Pai et al. are the first to report on a cholecystectomy transcolonically performed in a survival model [Pai R D, Fong D G, Bungda M E, et al., Transcolonic endoscopic cholecystectomy: a NOTES survival study in a porcine model. Gastrointest Endosc 2006; 64; pp. 428-434]. In their study performed with six pigs, Fong et al. also describe the transcolonic access into the peritoneal region [Fong D G, Pai R D, Thompson C C, Transcolonic endoscopic abdominal exploration: a NOTES survival study in a porcine model. Gastrointest Endosc 2007; 65; pp. 312-318]. However, both groups observe an increased bacterial contamination of the peritoneal region. Further transcolonic/transrectal studies have meanwhile been published, i.a., by McGee et al. [McGee M F, Marks J M, Chak A, et al., Feasibility of transrectal natural orifice transluminal endoscopic surgery (NOTES) in the porcine model. Poster presentation at the annual meeting of the Society of American Gastrointestinal and Endoscopic Surgeons (SAGES) 2007], Denk et al. [Denk P M, Whiteford M H, Swanstrom L L, TEM as a portal for NOTES. Poster presentation at the annual meeting of the Society of American Gastrointestinal and Endoscopic Surgeons (SAGES 2007] and Horton et al. [Horton K, Hathaway P, Krehel G, et al., New device for closure in transluminal surgery. Oral presentation at the annual meeting of the Society of American Gastrointestinal and Endoscopic Surgeons (SAGES) 2007].

According to estimates of the US Center for Disease Control, about 40 million people in the USA suffer from gastrointestinal tract disorders, which are of a high socioeconomic relevance on account of the direct and indirect disease costs of annually approximately 100 billion US$. Many of these patients could benefit from NOTES provided that the hypothetical advantages of NOTES over minimally invasive surgery can be also validated in human clinical studies in the short to medium term. In the current development phase, first of all the provision of appropriate instruments and methods as well as a landmark clarification of the still open questions with respect to the access path, infection risks, reliable closure, appropriate suture techniques and potential complications are of particular importance.

For transluminal surgery, normally a flexible surgical instrument (modified endoscope) is introduced into the abdominal cavity via an artificially provided orifice in the anterior gastric wall. Respective operations can then be performed in the peritoneal region with this flexible instrument (cholecystectomies, appendectomies, tubal ligations etc. have so far been experimentally performed). Upon termination of the operation and withdrawal of the instrument it is important to reliably close the entrance point into the abdomen in the anterior gastric wall.

Rectoscopes are generally known and described, for example, in patent specifications EP 0 858 283 and DE 199 35 725. Document EP 1 100 368 discloses a rigid sigmoidoscope including a disposable speculum comprising an essentially hollow tube defining an interior, an observation end and an insertion end, wherein the insertion end of the speculum is adapted for insertion into the anal canal of a patient, and coupling means adapted to allow a reusable light source to be connected to the observation end and to project light through the speculum into the bowel of the patient.

Trocar systems are known in various embodiments. They consist generally of a trocar tube as well as an obturator which is movable within the trocar tube and is provided at its distal end with a sharp, advantageously pyramidal tip or blade for piercing tissue. The trocar tube may be rigid or flexible.

The published patent application US 2005/0251190 discloses a trocar device for providing an access opening into a body cavity. Document US 2007/0088277 describes a surgical access port consisting of a trocar seal housing having at least one seal, a trocar cannula having a lumen sealed by the at least one seal, and a releasable connection system between the trocar cannula and the trocar housing.

Sealing devices are also known from, i.a., the documents U.S. Pat. No. 4,943,280, U.S. Pat. No. 4,655,752, U.S. Pat. No. 4,978,341 and EP 0 567 141. The latter relates to a valve system permitting introduction of surgical instruments into a patient's body, in particular via a trocar device. The maintenance of a gas seal is ensured for instruments of various sizes.

EP 0 630 213 discloses a trocar assembly with a retractable tip which can be moved from a cutting position to a shielded position. A further safety trocar is known from patent specification EP 0 604 197. The application EP 0 535 974 claims a trocar device consisting of a trocar tube defining an interior lumen and having an opening at its distal and proximal ends so that an elongate instrument having a smaller diameter can be moved within this trocar tube, characterized in that the trocar tube is made of a resiliently flexible sleeve member and has an inherent restoring force so that after the application of a bending force the trocar tube returns to its straight orientation.

EP 0 538 359 discloses a tube which can be introduced into the body through an orifice, comprises a bending mechanism and is provided which a manipulator for moving at least one pair of control cables. The patent specification DE 40 02 235 discloses a guide device for percutaneously introducing an endoscope with an insert body and a distal angle portion, characterized by a trocar consisting of an outer sheath and an obturator or grip portion extractably inserted in said outer sheath, and a guide tube to be inserted into said outer sheath in place of said obturator for forming a path of insertion. The guide tube is formed of a metal pipe and, seen in cross-section, its front end is rounded off in an arcuate shape.

The patent specification DE 43 12 147 discloses a trocar comprising an obturator having a piercing tip for piercing body tissue; a cannula for forming a passage in the tissue pierced by the piercing tip to enable the insertion of an endoscope or surgical tool into a body cavity, wherein the cannula consists of a flexible tube having an open proximal end; and a housing mounted on the open proximal end of the cannula and comprising a seal valve for sealingly guiding the obturator, characterized in that the housing is made of a soft material. The cannula is formed of a coil tube.

The published application DE 41 29 237 discloses a trocar sleeve through which auxiliary instruments can be passed, characterized in that at least part of its length is configured flexibly such that its shape adapts to any curvatures of rigid auxiliary instruments by elastic bending, but as to the rest without kinking. The trocar sleeve may comprise several longitudinal channels.

The document DE 28 20 239 shows a similar device. The published patent application US 2004/0044350 claims an articulated access sheath for providing an access to a body cavity consisting of a shaft having a proximal end, a distal end and a central lumen through which surgical instruments can be introduced into the body cavity. A portion of the shaft comprises a series of articulating members so that said portion of the shaft can be brought into a bent shape by at least one pullwire extending through at least one articulating member. Furthermore, a possibly flexible obturator is disclosed which can be positioned in the central lumen and supports a first shaping operation in the movable portion of the shaft.

As regards devices for the transluminal surgery, reference is made exemplarily to the following publications: the published application EP 1 602 336 claims a method and a device for providing a transmural access to the interior of a body cavity consisting of a flexible, multi-lumen overtube comprising a distal end and a main lumen configured to receive an endoscope and an outer lumen configured to receive an attachment mechanism configured to secure the distal end of the multi-lumen overtube to the organ wall. According to the respective European search report, i.a., the patent specification U.S. Pat. No. 6,030,365 is cited as a reference. Said US patent describes a device and method for creating sterile surgical access to internal body parts through a natural opening of an internal body cavity. The device is preferably inserted into a patient's rectum. In one possible embodiment, a concentric suction channel is provided in addition to a main access channel, said concentric suction channel extending in parallel to the main access channel.

Transgastric accesses to the abdominal cavity and respective methods are specifically mentioned, i.a., in the applications US 2004/0260245, US 2005/0148818 and US 2006/0237022.

### SUMMARY OF THE INVENTION

The invention is based on the problem of providing an improved trocar tube, trocar, obturator and/or rectoscope, in particular for use in the transluminal endoscopic surgery via natural body orifices of a human or an animal.

This problem is solved by the subject-matter according to the present claims.

The invention relates in particular to a trocar tube having a distal portion to be positioned at least in the region of or outside the body orifice, preferably in or at the rectum, and a proximal portion to be positioned within the human or the animal, wherein at least the proximal portion comprises an essentially non-deformable and/or non-bendable curvature. Non-deformable and/or non-bendable means that in use the trocar tube essentially maintains its shape, particularly its curvature, when used according to its purpose. The invention additionally or alternatively comprises an obturator for the trocar tube and/or a trocar according to the invention, wherein at least a proximal portion of a shaft of the obturator is made of a flexible and/or elastic material so that it can adapt to the curvature of the proximal portion of the trocar tube when inserted in the trocar tube.

The trocar is an instrument by means of which in surgery an opening is provided sharply or bluntly into a body cavity (e.g. peritoneal region, thoracic region) and kept open by a trocar tube. It is usually an obturator or pen which is arranged within the trocar tube having an inner diameter of, e.g., 0.5-12 mm and whose tip closes the aperture of the trocar tube. The trocar is introduced, for example, through the abdominal wall into the abdominal cavity. Upon withdrawal of the obturator from the trocar tube, the surgeon has the possibility of looking through the trocar tube into the abdominal cavity by means of an optical system (endoscope) or to perform minimally invasive surgery with graspers, scissors or other instruments within the abdominal cavity. This may be done through a trocar head which is distally connected to the trocar tube and comprises a gas-tight port for the aforementioned instruments. Modern trocars either consist of surgical steel or plastics and are manufactured as single-use or multiple-use instruments. The tip of the obturator is adapted to the trocar tube and preferably has one or more sharp cutting edges for the sharp preparation of the access path or has a blunt, conical tip for the blunt preparation. The trocar tubes specifically for the inspection of body and/or joint cavities (laparoscopy, thoracoscopy, arthroscopy) may comprise a valve mechanism and/or connection for insufflating (blowing in) CO₂ or rinsing fluids, or other supplementary equipment.

According to the prior art, the trocar tube is flexible and the obturator is inflexible in its non-straight shape. In contrast to that, the non-deformable and at least partially bent trocar tube according to the invention can be better steered and positioned and a respective obturator can be better steered in order to carry out the perforation of the abdomen, in particular the anterior rectal wall, in a well directed and precise manner. Preferably, the trocar tube is long enough to enable access into the abdominal cavity and where necessary the aforementioned exemplary surgical operations.

It is preferred that the entire trocar tube is essentially non-deformable. Preferably, it is made of an easily sterilizable material, such as, for example, of an appropriate plastic material, metal, etc. A plastic material is preferred for reasons of costs and weight. The trocar tube according to the invention is preferably packaged individually or as a set together with the further described and claimed elements as a disposable article or after sterilization.

In the preferred trocar tube according to the invention, the curvature of the proximal portion is essentially constant. However, the trocar tube may also have an increasing or decreasing radius or several radii towards its free end. The shape is predeterminable according to the anatomic requirements and other operation-related conditions. A preferred curvature radius of a middle axis of the proximal portion of the trocar tube is about 45-75 mm, preferably about 50-70 mm, further preferably about 55-65 mm and further preferably about 60 mm.

A trocar tube according to the invention preferably has an inner diameter of about 2-22 mm, preferably of about 10-20 mm, further preferably of about 14-18 mm, further preferably of about 15-17 mm and further preferably of about 16 mm, over an at least substantial part of its entire length.

Further preferably, a trocar tube has a wall thickness of about 0.2-2 mm, preferably of about 0.8-1.2 mm, further preferably of about 0.9-1.1 mm, further preferably of about 1 mm, over an at least substantial part of its entire length. Most preferably, the entire trocar tube has this wall thickness with the only exception of the fixing means for a trocar head at the distal end and a taper at the proximal end as mentioned hereinafter.

The trocar tube according to the invention has an entire length of about 230-400 mm, preferably of about 280-370 mm, further preferably of about 300-350 mm, further preferably of about 315-335 mm and further preferably of about 325 mm. The length of the trocar is configured such that it can be introduced up into the abdominal cavity and freely manipulated in the body. The trocar provides a fixing possibility for being attachable at a holder or manipulator. The bent front section of the trocar has a ski-like shape because of the curved configuration of the rectosigmoid. It is thereby more easily possible to pierce the rectum. Additionally, the endoscope can be prepositioned at the site of operation on account of the bent shape of the trocar.

Preferably, the effective length of a straight section of the trocar tube is about 180-300 mm, preferably about 200-280 mm, further preferably about 220-260 mm, further preferably abut 232-252 mm, further preferably about 238-247 mm and further preferably about 242 mm. Apart from the effective length, the entire length is made up of the length of the bent section and the length of an area for connection to the trocar head.

According to the invention, the absolute total deflection of the trocar tube in the area of the curvature of the proximal portion towards one side is about 15-18 mm, preferably about 16-17 mm, further preferably about 16.5 mm. What is measured in this connection is the distance parallel to the adjacent outer wall of the trocar tube in its straight section up to the outermost point of the proximal section, preferably at its free end. Alternatively, it is also possible to measure the maximum angle between a longitudinal axis of the non-bent or straight section of the trocar tube and the longitudinal axis or its tangent of the most bent proximal section. This maximum angle is about 20-70°, preferably about 30-60°, further preferably about 35-55°, further preferably about 40-50° and further preferably about 45°. With respect to the last mentioned case and with the curvature having the preferred uniform configuration, this means that the bent section describes ⅛ of a full circular arc. However, ⅓ or ¼ of a full circular arc would also be conceivable for specific purposes.

The aforementioned dimensions are particularly appropriate for the present use via the rectosigmoidal transition into the abdomen.

Preferably, a free end of the proximal portion is tapered. It is preferred that at least the proximal portion, preferably the entire trocar tube, has an essentially uniform cross-section that is preferably oval or circular or round. A circular cross-section is less complex but an oval cross-section is advantageous in that an obturator according to the invention, which will be described hereinafter, can be guided in a torsion-proof way when, for example, the shape and positioning of its sharp tip are of particular importance. Besides, an oval cross-section provides some more room to move for a later introduced endoscope.

Further according to the invention, the trocar tube comprises a means for the liquid-tight, preferably gas-tight connection to a trocar head. The connection means preferably has a radial bulging so that a respective device at the trocar head can elastically engage behind said bulging in an interlocking manner.

Furthermore, the trocar tube preferably has external markings for the purpose of providing readings of the radial orientation and/or its inserted length. The surgeon is thereby given help for positioning and if necessary further steering the trocar tube.

According to the invention, the trocar tube may further comprise at least one working channel which is preferably arranged externally at the outer wall of the trocar tube. Any auxiliary means, equipment etc. can also be passed from outside to the site of perforation and/or into the abdominal cavity in a well directed manner through such a working channel.

Preferably, a trocar according to the invention comprises a trocar tube and a trocar head. These two components may be provided or aseptically packaged integrally, individually or together in a kit.

It is preferred that the trocar tube and the trocar head can be connected to each other in a liquid-tight and preferably gas-tight manner by providing a seal and/or a positive connection in between or they are already configured integrally. This seal may be also present in the aforementioned kit.

According to the invention, the trocar tube and the trocar head can be releasably connected to each other. This would enable a modular composition for individually assembling the most appropriate components and/or the individual configuration of one of the components as a disposable article. The trocar head is usually relatively complex and, therefore, in the latter case it would be possible to offer only the trocar tube as a disposable article made of an appropriate plastic material.

The trocar head is preferably provided with at least one gas-tight valve and/or at least one inlet and/or outlet for gas(es) and/or liquid(s), for example for insufflating gas or discharging taurolidine solution.

Furthermore, the trocar preferably comprises a means for fixing the trocar, preferably to a holder or manipulator.

The aforementioned obturator according to the invention comprises a tip which is tapered such that it essentially corresponds to the taper of the free end of the proximal portion of the obturator for the purpose of better perforating the intestinal wall. Alternatively or additionally, the trocar and/or trocar tube according to the invention can also be used or packaged in a kit with other surgical tools.

A controlled introduction of the obturator is preferred in order that the tissue surrounding the body orifice (for example the large intestine) is not unintentionally injured by the obturator during the introduction of the trocar and/or in order that the preferably sharp and preferably tapered or conical tip of the obturator is not damaged. During the insertion of the trocar, the conical tip of the obturator can be positioned preferably so as to be withdrawn in the trocar tube and/or the conical tip of the obturator is retractable into the trocar tube so that it does not project towards the front beyond the trocar tube. This means that the obturator is in a withdrawn position. After the trocar has been steered to the piercing site, the perforation of the intestinal wall can be performed (preferably under visual control) by moving or pushing the obturator out of the trocar tube. By pushing the obturator out of the trocar tube, the obturator tip projects beyond the proximal end of the trocar tube so that the tip can be brought into engagement with the surrounding tissue, i.e. the obturator is in an extended position.

The trocar preferably comprises a retaining means at its distal end in order to maintain the obturator in the withdrawn position and/or to preload it into the withdrawn position. The retaining means preferably comprises at least one springy element, for example one or more pressure springs, which preloads the obturator into the withdrawn position. When a force is exerted which counteracts the springy element, the obturator is pushed forwards against the preload (i.e. in the proximal direction) so that the tip projects from the trocar tube so that the surrounding tissue, for example the intestinal wall, can be pierced. After releasing the obturator, the obturator with the conical tip is withdrawn into the initial position, i.e. the withdrawn position, by means of the retaining means. Preferably, the retaining means comprises limit stops between which the obturator can be moved to and fro from a withdrawn position to an extended position along a predetermined length. A first limit stop can be configured such that the tip of the obturator is at the level of the proximal end of the trocar or deeper within the trocar, i.e. the obturator is in a withdrawn safe position. A second limit stop can be configured such that the entire tip of the obturator extends beyond the proximal end of the trocar. Preferably, the second limit stop is configured such that the obturator tip rests flush at the tapered proximal end of the trocar tube.

The tip of the obturator has a maximum outer diameter of about 2-21 mm, preferably of about 11-19 mm, further preferably of about 13-17 mm, further preferably of about 14-16 mm and further preferably of about 15 mm. Further preferably, a shaft of the obturator arranged behind the tip of the obturator has a maximum outer diameter of about 6-19 mm, preferably of about 8-17 mm, further preferably of about 10-15, further preferably of about 11-14 mm and further preferably of about 12 mm.

Preferably, the trocar, i.e. the trocar tube and the trocar head and the tip of the completely introduced obturator, has an entire length of about 280-420 mm, preferably of about 300-400 mm, preferably of about 320-380 mm, further preferably of about 340-360 mm and further preferably of about 350 mm.

The invention also relates to a rectoscope adapted to the aforementioned elements, which in particular comprises a port for the trocar and the port is configured such that, when the trocar is inserted, it enables a tilting movement of the trocar for piercing the intestinal wall. Typically, a rectoscope is used for performing an endoscopy of the straight intestine (rectoscopy). A rectoscopy is performed for examining the last portion of the colon in front of the after and mostly also of the last few centimetres of the rectum.

The rectoscope preferably has a tube shaft whose proximal end comprises a chamfer. This chamfer and the port are aligned such that they enable and/or further support the tilting movement of the trocar or for a start enable the obturator tip to perforate the respectively exposed intestinal wall. An optical system which has preferably been introduced in the vicinity of the perforation site and can be looked through from the outside can then render the obturator tip and the perforation observable. Preferably, the chamfer has an angle towards the middle axis of the tube shaft of about 45°-80°, preferably of about 60°-80°, further preferably of about 65°-75° and further preferably of about 70°.

The rectoscope preferably comprises a tube shaft and at least two different caps, preferably with a quick closure for distally closing the tube shaft in a gas-tight manner. One of the caps can be provided with the port for the trocar and the other cap with the port for introducing further operation instruments. Alternatively, only one cap with ports for all equipment to be used and for the trocar may be provided.

According to the invention, the rectoscope has an entire length of about 150-250 mm, preferably of about 170-230 mm, further preferably of about 180-220 mm, further preferably of about 190-210 mm and further preferably of about 200 mm.

As already mentioned, the invention also relates to a set or several sets comprising at least two elements of a trocar tube, a trocar, an obturator and/or a rectoscope. A set comprising all these elements and further sets for the subsequent use comprising only a trocar tube and an obturator and perhaps comprising a trocar head would be conceivable.

The invention in particular relates to the use of all elements described and claimed above and below for the transluminal endoscopic surgery and/or diagnosis via natural body orifices.

The invention also relates to a method for preparing a treatment, for the treatment and/or diagnosis in the transluminal endoscopic surgery via natural body orifices of a human or an animal. Such a method comprises the steps of providing a trocar tube, a trocar, an obturator and/or a rectoscope and as the case may be the respective surgical operation.

The field of application of the new invention is explained using the example of an intervention. The intra-abdominal lumen of the rectosigmoidal transition is easily accessible via a specific rectoscope. A tobacco pouch suture is made at the level of the rectosigmoidal transition. Under direct vision, the endoscopic trocar comprising the introduced obturator is subsequently introduced into the rectoscope and the anterior rectal wall is precisely pierced to enable access into the abdominal cavity. Upon removal of the obturator, the actual flexible operation instrument can be introduced into the abdominal wall. Upon termination of the intra-abdominal procedure, the flexible operation instrument and the trocar are removed and the tobacco pouch suture is closed through the rectoscope.

Preferably, the perforation of the intestinal wall is made under ultrasonic control in order to avoid that the bladder is injured when performing the piercing step with the trocar. Prior to this delicate piercing process with the obturator and preferably after the introduction of the rectoscope and after making the tobacco pouch suture, the intended piercing site is set with a puncture needle comprising a needle wire. Thereafter, the trocar comprising the obturator can be advanced to the piercing point via the needle wire and subsequently, the intestinal wall can be perforated at the intended site.

This preferably controlled procedure is preferably performed under direct vision using an endoscopic ultrasound probe which is also introduced into the rectoscope. It can thereby be avoided that additional surrounding organs such as, e.g., a loop of the small intestine are injured. Preferably an endoscopic ultrasound head or probe is introduced through an instrument channel of the rectoscope. A puncture needle can be introduced through a further instrument channel by means of a specific, preferably proximally tapered and preferably bent guide instrument whereby the intestinal wall can be pierced at the correct site through the tobacco pouch suture. The guide instrument is subsequently removed. The obturator, preferably together with the trocar, can be advanced via and/or along the introduced and preferably remaining needle wire.

The puncture needle and/or the needle wire has preferably a diameter of about 0.5-3 mm. The guide instrument can be made of different materials, for example plastic material, stainless steel, shape memory materials, etc. At its front (proximal) part, the guide instrument preferably has a small diameter, for example of 1-4 mm. Moreover, at its proximal end, the guide element preferably comprises a bent section which preferably has a short length, preferably of about 10-40 mm, for reasons of strength and stability. The back (distal) part of the guide instrument preferably has a larger diameter, for example of about 3-7 mm, preferably of 5 mm. According to preferred embodiments, the guide instrument has a wall thickness of at least 1 mm at least at its back end. Besides, the guide instrument can be more easily introduced and operated by means of an optional distal handle. Preferably, the guide instrument comprises markings along its longitudinal direction so that the penetration depth of the instrument can be easily checked.

In further support of the piercing process, the preferably flexible or semi-rigid obturator may comprise a central insertion or guide channel for the puncture needle. A further improved, controlled piercing operation or perforation operation can be performed with the puncture needle through this guide channel under visual control. The front (proximal) part of this guide channel is preferably adapted to the diameter of the needle and the back (distal) part thereof preferably has a diameter of about 2-4 mm.

The present invention enables a safe, sterile, sigmoidal access to the peritoneal region. Using taurolidine solution—a substance being successfully used since 1975 in the septic abdominal surgery and since 1980 in the traumatology as a surgical rinsing solution for the treatment of infections and having antibacterial and fungicidal effect while not having any toxic short-term or long-term effects—during first examinations with a porcine model did not reveal any complications due to contamination. At first, preferably a protective fluid is introduced into the abdominal cavity in a manner known per se, preferably via a Verres needle. Subsequently, preferably about 1 l Taurolin solution (Taurolin, Boehringer Ingelheim, Ingelheim, Germany) and preferably about 2.5 l Ringer's solution are introduced via this needle. In the test, pigs were put into the so-called anti-Trendelenburg position (head at 30° to the top). Since the injected fluid mainly concentrates in the lesser pelvis, the movable and gas-comprising organs etc. in the pelvis are removed from the pelvic floor and the rectosigmoid. In this way, the area to be later perforated is advantageously exposed. Thus, according to the invention, the rectosigmoidal transition is exposed when the patient is correctly positioned so that the area to be perforated remains as free from organs etc. as possible and a procedure which is safe for the patient is rendered possible.

It is a great advantage of the invention to ensure a sterile safe access into the abdomen via the rectosigmoidal transition by means of an appropriate apparatus system and additionally to render possible a reliable closure of the access site after termination of the intervention. In particular the manoeuvrability of the trocar or the trocar tube for the purpose of positioning the obturator in a well-directed way is considerably more advantageously possible than in the prior art.

A further advantage of the invention is that a surgical instrument and method are provided which afford a sterile access into the abdominal cavity via the rectosigmoidal region and permit a safe closure of the access orifice at the end of the intervention.

### BRIEF DESCRIPTION OF THE DRAWINGS

Advantageous embodiments of the invention are exemplarily shown in the Figures, in which:

FIG. 1 shows a perspective and schematic illustration of a trocar with inserted obturator according to the invention,

FIG. 2 shows a cross-section through a trocar with inserted obturator according to the invention,

FIG. 3 shows a cross-section through a rectoscope according to the invention,

FIG. 4 shows a perspective and schematic illustration of a rectoscope with inserted endoscopic trocar and obturator according to the invention,

FIG. 5 shows a cross-section through a trocar with inserted obturator according to a further embodiment according to the invention,

FIG. 6 shows a cross-section through a rectoscope with inserted guide instrument and inserted ultrasound probe according to the invention.

### DETAILED DESCRIPTION

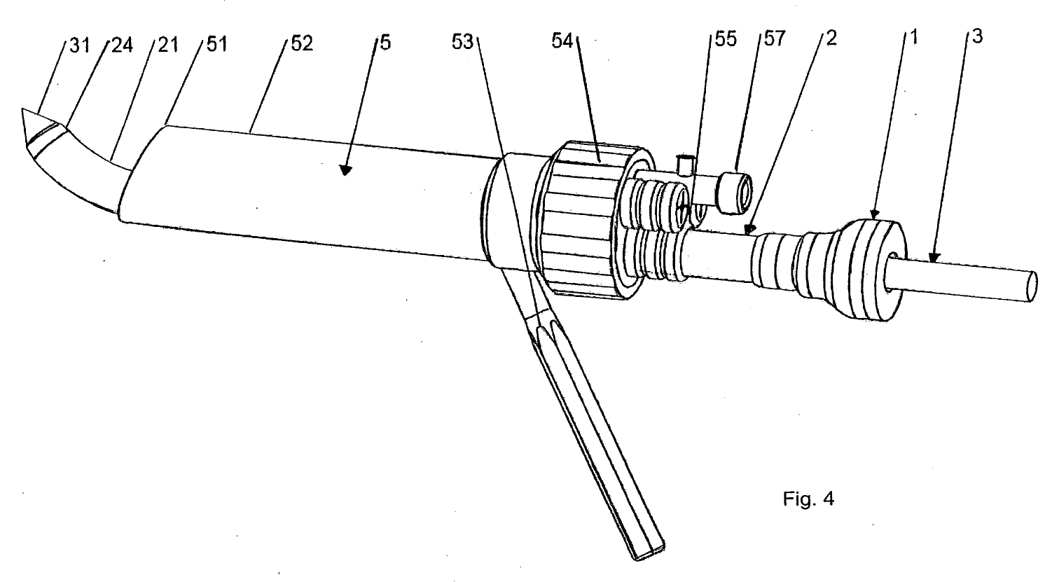

FIG. 4 shows best the common arrangement or mode of operation of the elements trocar, obturator and rectoscope according to the invention. A rectoscope 5 accommodates a trocar tube 2 and an obturator 3. These elements are further illustrated in the other Figures. A likewise preferably used trocar head is not shown in FIG. 4.

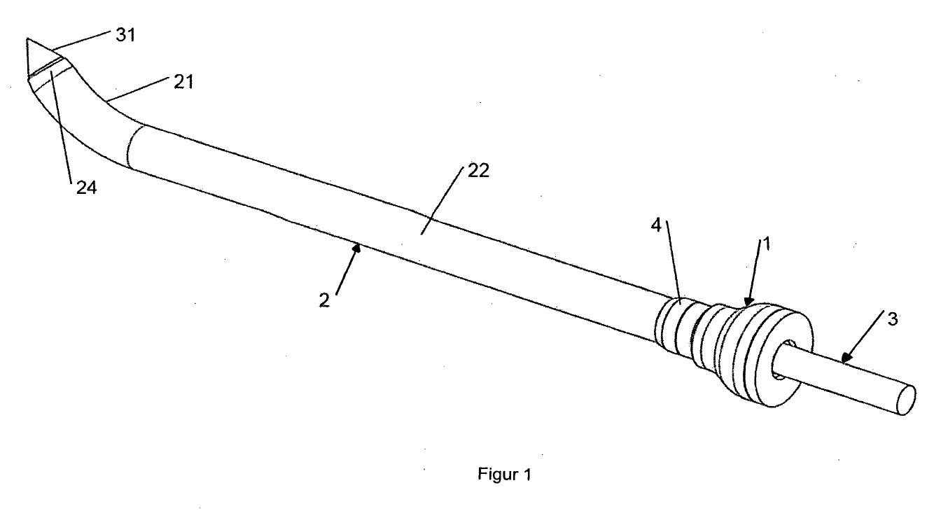

FIG. 1 shows mainly an arrangement of the trocar tube 2 according to the invention with the trocar head 1 arranged on it. A seal 4 is also shown, which is preferably arranged for providing a gas-tight sealing between the trocar tube 2 and the trocar head 1 and may also represent an elastic intermediate layer for providing better releasability of these elements. The obturator 3 having a tip 31 is also shown and extends through the trocar tube 2 and the trocar head 1. The trocar tube 2 has a bent section 21 and may comprise at least one straight or unbent section 22.

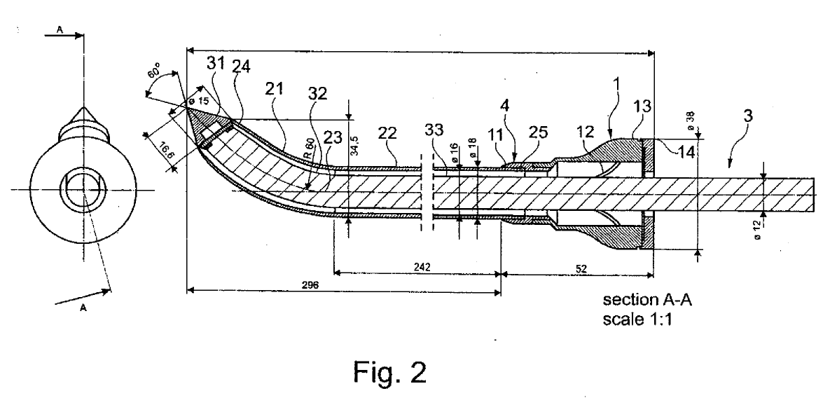

FIG. 2 is a cross-sectional view illustrating the cooperation of these elements. At its distal end, the trocar tube with its bent and its straight sections 21, 22 is connected to the trocar head 1 via a seal 4. The trocar head 1 has a body 13 and is shown in a preferred embodiment comprising a bulging at the trocar tube 2 which is encompassed by a respective recess at he trocar head 1 in a manner known per se. The seal is shown in a portion between the trocar head 1 and the trocar tube 2 outside of this connection between these elements in a region in which a neck 11 extends at the trocar head 1 further beyond the trocar tube 2. Other known connections, such as particularly click, bayonet, screw connections etc. are also appropriate, in particular if they can be configured in a gas-tight manner. Due to the shown embodiment, the arrangement is additionally secured against rotation.

The trocar head 1 additionally comprises a valve 12 which gas-tightly seals a shaft 33 of the obturator 3. Furthermore, a lid 14 is provided which like the valve 12 permits the passage of the larger tip 31 but above all is provided as a mechanical protection.

The trocar tube 2 is provided with a straight section 22 arranged proximally to the trocar head and followed by a bent section 21. Alternatively—depending on the anatomic conditions or further special features—it is also possible that the sections are arranged in another order or that there are several straight and/or bent sections 21, 22. In the illustrated embodiment, however, a preferred arrangement is depicted which is anatomically and ergonomically advantageous, specifically for the aforementioned use.

The bent section 21 comprises a lateral deflection which is indicated in the depicted dimension together with the outer diameter (totalling 34.5 mm). When the most preferred outer diameter of 18 mm is subtracted therefrom, a maximum deflection of 16.5 mm is obtained.

The radius R60 shown in FIG. 2 relates to the middle line 23 of the trocar tube 2.

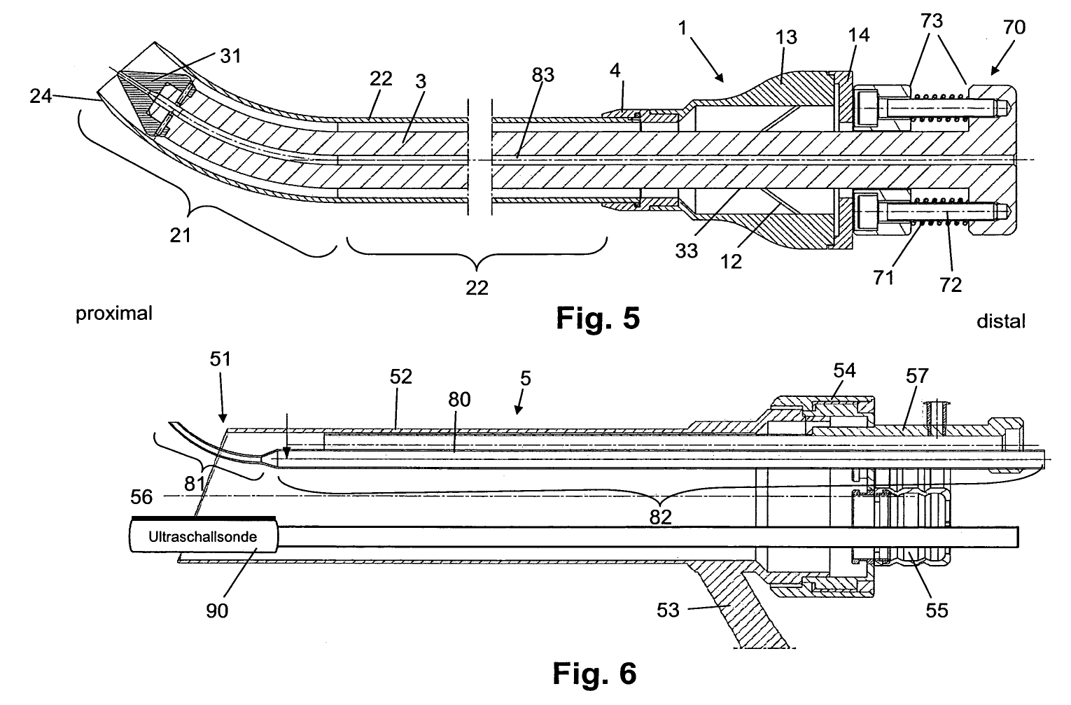

At the front (proximal) end of the trocar tube 2, the tapered end 24 can be seen whose taper is advantageously continued at the tip 31 of the obturator 3 when the obturator is extended. This preferably sharp tip 31 is mounted on the shaft 32, 33 in an appropriate manner, preferably by a pin. For the purpose that, for example, the large intestine is not unintentionally injured by the tip 31 of the obturator during the introduction of the trocar and/or the preferably sharp and preferably tapered or conical tip 31 of the obturator is not damaged, a retaining member 70 can be provided at the distal (back) end of the trocar (see FIG. 5). FIG. 5 shows an embodiment that is very similar to that of FIG. 2, but with an additional retaining member 70. It is preferred that the conical tip 31 of the obturator can be positioned so as to be withdrawn or that it can be concealed during the introduction of the trocar into the trocar tube so that the tip of the obturator does not project beyond the front end of the trocar tube. This is achieved by the retaining member 70. The retaining member 70 preferably comprises at least one spring mechanism 71, 72 with at least one springy element 71 (for example with one or more pressure springs 71) which preloads the obturator into its withdrawn position. In the embodiment shown in FIG. 5, the springs 71 are guided on the shafts 72 (for example with one or more cylinder screws). After the trocar has been advanced to the desired piercing site, the perforation of the intestinal wall can be performed by moving or pushing the obturator out of the trocar tube. To this end, a force counteracting the springy element so that the obturator is pushed forward (i.e. into the proximal direction) against the preload and the tip 31 projects from the trocar tube in order that the intestinal wall can be pierced and/or perforated. When the exertion of force is discontinued, the obturator with its conical tip 31 is withdrawn into its initial position. Preferably, the retaining member comprises limit stops 73 between which the obturator can be moved to and fro from a withdrawn position into an extended position. The length along which the obturator can be displaced can be defined by the appropriate selection of the limit stops. A first limit stop is configured, for example, such that the tip of the obturator is at the level of the proximal end of the trocar or deeper in the trocar tube, i.e. the obturator is in its withdrawn position. This can be achieved, for example, by an appropriate length of the shafts 72. A second limit stop 73 can be configured such that the tip 31 of the obturator projects from the proximal end of the trocar. Preferably, this trocar is configured such that the obturator tip rests flush at the tapered proximal end 24 of the trocar tube 24.

At least one section 32 of the shaft of the obturator 3 is configured flexibly in order to be able to adapt to the curved section 21 of the trocar tube 2. Additionally, it may be configured elastically, but a plastically deformable variant is also conceivable. However, it should be not or only little compressible in order that the tip 31 can be moved as dependently on the movement of the opposite end as possible. A further section 33 is arranged at the flexible section 32 which does not necessarily have to be configured integrally with it and actually may be rigid or at least not elastic. In a preferred embodiment, however, the entire shaft 32, 33 is made of a flexible material.

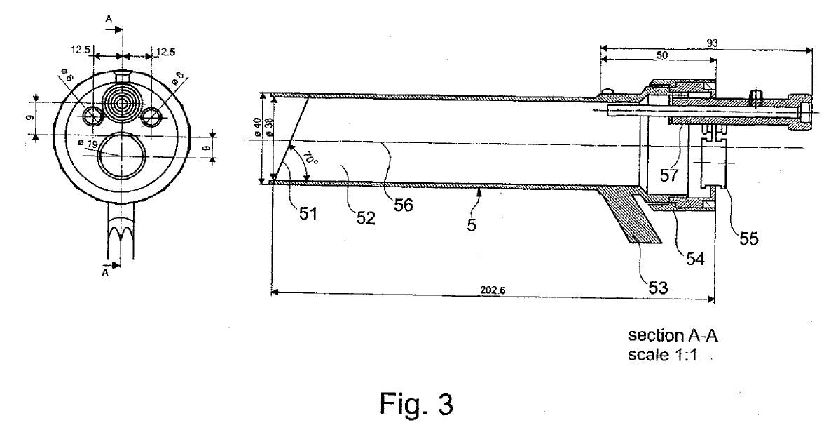

FIGS. 3 and 4 show an embodiment of a rectoscope 5 which is particularly appropriate for the above described trocar (trocar tube 2 with inserted obturator 3 and trocar head 1 in FIG. 4). The rectoscope 5 consists of a tube shaft 52 with a longitudinal axis 56 and a cap 54. Both elements are preferably gas-tightly connected by means of a quick closure. A handle 53 supports the surgeon in manipulating and positioning the rectoscope. A port 55 is provided for receiving and gas-tightly sealing the trocar tube 2. In a particularly advantageous way for the use according to the invention, the port 55 permits that the trocar and/or trocar tube 2 can be swivelled or tilted to move the tip 31 shown again in FIG. 4 and to perforate the intestinal wall. The chamfer 51 at the proximal end of the rectoscope 5, more precisely the tube shaft 52, is also clearly to be seen. This chamfer 51 improves the exposure of the portion of the intestine to be perforated and is preferably arranged relative to the port 55 such that the part of the tube shaft further exposed by the chamfer 51 is diametrically opposite to the port 55 with respect to the longitudinal axis 56 (cf. FIGS. 3 and 6).

Furthermore, FIG. 3 shows an optical channel 57 depicted in a shortened way. It can be seen particularly in FIGS. 3, 4 and 6 that the port 55, the optical channel 57 and if necessary any further inlets are arranged in or at the cap 54, which preferably are gas-tight. Preferably two different caps are necessary for performing the operation through the rectoscope 5. The tobacco pouch suture and microsurgical interventions necessary for the operation are performed through the first cap consisting of three insertion channels for surgical instruments. The second cap also consists of three insertion channels. This cap is mounted after having made the tobacco pouch suture and permits the insertion of the endoscopic trocar. The access channels for instruments are provided with valves to ensure the gas-tightness. The optical system is introduced through a special channel and, depending on the intervention, permits the exposure of the surgical field. The optical system is additionally secured and sealed by means of a quick closure.

According to a preferred embodiment, the perforation of the intestinal wall is performed by means of an obturator under ultrasonic control in order to avoid, for example, that the bladder is injured when performing the piercing step with the trocar. Prior to this delicate piercing process with the obturator and preferably after the introduction of the rectoscope 5 and after making the tobacco pouch suture, the intended piercing site is set with a puncture needle and/or a puncture wire. The puncture needle and/or puncture wire is preferably guided through a wire guide instrument or guide instrument 80 which can be inserted into the rectoscope (see FIG. 6). Preferably the guide instrument 80 is inserted into the rectoscope via one of the aforementioned instrument channels 57. The guide instrument 80 preferably comprises a proximally tapered and preferably bent section 81 for guiding the puncture needle and/or puncture wire to the correct site through the tobacco pouch suture in the intestinal wall. This procedure is preferably carried out under ultrasonic control. To this end, for example, an endoscopic ultrasound head or ultrasound probe 90 can be introduced through the lower large insertion channel 55 of the rectoscope cap. The guide instrument 80 is subsequently removed, wherein the wire remains in the rectoscope. Thereafter, the trocar comprising the obturator can be advanced via the introduced needle wire, i.e. the needle wire serves as a guide for the obturator. The trocar comprising the obturator 3 is advanced to the piercing point via the needle wire so that subsequently the intestinal wall can be perforated at the correct site. By means of this preferred controlled procedure under direct vision by means of the endoscopic ultrasound probe 90, it is possible to avoid that additional surrounding organs such as, e.g., a loop of the small intestine are injured.

A puncture needle preferably has a diameter of about 0.5-3 mm. The guide instrument 80 can be made of different materials, such as, for example, plastic material, stainless steel, shape memory materials, etc. At its front part 81, the guide instrument 80 preferably has a small diameter, for example of 1-4 mm. The front section 81 is preferably bent and, for reasons of strength and stability, preferably has a short length, preferably of about 10-40 mm. The back part 82 of the instrument 80 preferably has a larger diameter, for example of about 3-7 mm, preferably of 5 mm. According to preferred embodiments, the guide instrument 80 has a wall thickness of at least 1 mm at least at its back end. The instrument can be more easily introduced and operated by means of an optional distal handle (not illustrated). Preferably, the instrument comprises markings along its longitudinal direction so that the penetration depth of the instrument 80 can be easily checked.

In further support of the piercing process, the preferably flexible or semi-rigid obturator may comprise a central insertion or guide channel 83 (see FIG. 5) for the puncture needle. A further improved, controlled piercing operation can be performed with the puncture needle through this guide channel 83 under visual control. The front part of this guide channel 83 is preferably adapted to the diameter of the needle and the back part thereof preferably has a diameter of about 2-4 mm.

By means of external markings at the trocar tube, which are not illustrated, the radial orientation of the trocar can be read. The markings are used in order to align the curved section and/or the bent front part of the tube. By means of additional markings, it is also possible to read the length of the trocar that is currently in the body.

The invention also comprises individual features in the Figures even though they are illustrated therein in connection with other features and/or are not mentioned above or below.

The invention also encompasses embodiments comprising any combination of features which are mentioned or shown in connection with different embodiments above or below.

The invention likewise encompasses the precise or exact terms, features, numerical values or ranges, etc. when these terms, features, numerical values or ranges are mentioned above or below in connection with terms such as, e.g., “about, approximately, around, essentially, generally, at least, not less than” etc. (i.e. “about 3” is also meant to comprise “3” or “essentially radial” is also meant to comprise “radial”).

What is claimed is:

1. A trocar tube for use in transluminal endoscopic surgery via a natural body orifice of a human or an animal, the trocar tube comprising:
a distal portion (22) to be positioned at least in the region of or outside the natural body orifice; and
a proximal portion (21) to be positioned within the human or animal, wherein:
at least the proximal portion (21) comprises at least partially an essentially non-deformable curvature.

2. The trocar tube according to claim 1, wherein the entire trocar tube (2) is essentially non-deformable.

3. The trocar tube according to claim 1, wherein the curvature of the proximal portion (21) is essentially constant and has a curvature radius at a middle axis of the proximal portion (21) of about 45-75 mm.

4. The trocar tube according to claim 1, wherein the trocar tube (2) has an inner diameter of about 2-22 mm over an at least substantial part of its entire length.

5. The trocar tube according to claim 1, wherein the trocar tube (2) has a wall thickness of about 0.2-2 mm over at least a substantial part of its entire length.

6. The trocar tube according to claim 1, wherein the trocar tube (2) has an entire length of about 230-400 mm.

7. The trocar tube according to claim 1, wherein the effective length of a straight section of the trocar tube is about 180-300 mm.

8. The trocar tube according to claim 1, wherein:
the proximal portion (21) has an absolute total deflection towards one side of about 15-18 mm; and/or
the maximum angle between a longitudinal axis of a non-bent section of the trocar tube and the longitudinal axis of the most bent proximal section is about 20-70°.

9. The trocar tube according to claim 1, wherein a free end (24) of the proximal portion (21) is tapered.

10. The trocar tube according to claim 1, wherein at least the proximal portion (21) has an essentially uniform cross-section that is oval or round.

11. The trocar tube according to claim 1, further comprising connection means (25) for the liquid-tight and/or gas-tight connection to a trocar head.

12. The trocar tube according to claim 11, wherein the connection means (25) is a radial bulging to enable elastic engagement behind said bulging in an interlocking manner.

13. The trocar tube according to claim 1, wherein the trocar tube (2) externally has markings for providing readings of the radial orientation and/or its inserted length.

14. The trocar tube according to claim 1, wherein the trocar tube (2) externally has at least one working channel.

15. A trocar comprising:
a trocar tube (2) for use in the transluminal endoscopic surgery via a natural body orifice of a human or an animal, the trocar tube comprising: a distal portion (22) to be positioned at least in the region of or outside the natural body orifice and a proximal portion (21) to be positioned within the human or animal, wherein at least the proximal portion (21) comprises at least partially an essentially non-deformable curvature; and
a trocar head (1) connected to a proximal end of the trocar tube (2).

16. The trocar according to claim 15, wherein:
the trocar tube (2) and the trocar head (1) are connected to each other in a liquid-tight and/or gas-tight manner by providing a seal (4) in between.

17. The trocar according to claim 15, wherein:
the trocar tube (2) and the trocar head (1) are releasably connected to each other.

18. The trocar according to claim 15, wherein:
the trocar head (1) is provided with at least one gas-tight valve (12).

19. The trocar according to claim 15, wherein:
the trocar head (1) comprises at least one inlet and/or outlet for gas(es) and/or liquid(s).

20. The trocar according to claim 15, further comprising:
a trocar head body (13) for fixing the trocar to a holder or manipulator.

21. A trocar tube (2) in combination with an obturator (3), wherein:
the trocar tube is for use in transluminal endoscopic surgery via a natural body orifice of a human or an animal, and comprises:
a distal portion (22) to be positioned at least in the region of or outside the natural body orifice; and
a proximal portion (21) to be positioned within the human or animal, wherein:
at least the proximal portion (21) comprises at least partially an essentially non-deformable curvature; and
the obturator comprises a shaft having at least one proximal section (32) made of a flexible and compressible material so that it can adapt to the curvature of the proximal portion (21) of the trocar tube (2) when being introduced thereinto, said at least one proximal section (32) configured to undergo essentially no compression upon being introduced into the trocar tube.

22. The trocar tube (2) in combination with the obturator (3) according to claim 21, wherein
the obturator (3) comprises a tip (31) having a taper corresponding to the taper of the free end (24) of the proximal portion (21) of the trocar tube (2) to facilitate piercing an intestinal wall.

23. The trocar tube (2) in combination with the obturator (3) according to claim 21, wherein:
the tip (31) of the obturator (3) has a maximum outer diameter of about 2-21 mm.

24. The trocar tube (2) in combination with the obturator (3) according to claim 21, wherein a shaft (32) of the obturator arranged behind the tip (31) of the obturator (3) has a maximum outer diameter of about 6-19 mm.

25. The trocar tube (2) in combination with the obturator (3) according to claim 21, wherein the obturator (3) comprises a guide channel (83) configured essentially along the axial direction of the obturator in order to guide the obturator along a guide wire.

26. A system comprising:
a trocar comprising:
a trocar tube (2) for use in the transluminal endoscopic surgery via a natural body orifice of a human or an animal, the trocar tube comprising: a distal portion (22) to be positioned at least in the region of or outside the natural body orifice and a proximal portion (21) to be positioned within the human or animal, wherein at least the proximal portion (21) comprises at least partially an essentially non-deformable curvature; and
a trocar head (1) connected to a proximal end of the trocar tube (2); and
an obturator (3) configured to be received into the trocar tube (2) and comprising:
a tip (31) and shaft (33) having at least one proximal section (32) made of a flexible and compressible material so that it can adapt to the curvature of the proximal portion (21) of the trocar tube (2) when being introduced thereinto, said at least one proximal section (32) configured to undergo essentially no compression upon being introduced into the trocar tube;
wherein:
when the obturator occupies the trocar tube (2), the system has a length of about 280-420 mm.

27. The system according to claim 26, wherein:
the obturator (3) is attached to the trocar by a retaining member (70) which preloads the obturator into a withdrawn position in order that the tip (31) of the obturator rests in a protected region within the trocar.

28. The system according to claim 27, wherein:
the retaining member (70) comprises a spring mechanism having at least one spring (71) for preloading.

29. A rectoscope (5) having incorporated therewith a trocar comprising:
a trocar tube (2) for use in the transluminal endoscopic surgery via a natural body orifice of a human or an animal, the trocar tube comprising: a distal portion (22) to be positioned at least in the region of or outside the natural body orifice and a proximal portion (21) to be positioned within the human or animal, wherein at least the proximal portion (21) comprises at least partially an essentially non-deformable curvature; and
a trocar head (1) connected to a proximal end of the trocar tube (2); wherein:
the rectoscope (5) comprises a port (55) into which the trocar is receivable, the port (55) being configured to enable a tilting movement of the trocar, a piercing of the intestinal wall, or both.

30. The rectoscope (5) according to claim 29, comprising:
a tube shaft (52) whose proximal end (51) comprises a chamfer and is aligned such that the chamfer further supports the tilting movement of the trocar.

31. The rectoscope (5) according to claim 30, wherein:
the chamfer of the proximal end (51) has an angle towards a middle axis (57) of the tube shaft of about 45°-80°.

32. The rectoscope (5) according to claim 29, comprising:
a tube shaft (52) and at least two different caps provided with a quick closure for distally closing the tube shaft (52) in a gas-tight manner.

33. The rectoscope (5) according to claim 29, having a length of about 150-250 mm.

34. A set comprising at least two of the following elements:
(a) a trocar tube (2) comprising:
a distal portion (22) to be positioned at least in the region of or outside a natural body orifice of a human or an animal; and
a proximal portion (21) to be positioned within the human or animal, wherein:
at least the proximal portion (21) comprises at least partially an essentially non-deformable curvature;
(b) a trocar comprising the trocar tube and a trocar head;
(c) an obturator (3) receivable into the trocar tube; and
(d) a rectoscope (5) having a port (55) into which the trocar is receivable.

35. A method for treatment and/or diagnosis of a human or an animal entailing transluminal endoscopic surgery via a natural body orifice of said human or animal, comprising the steps of:
introducing a taurolidine solution into the abdominal cavity of said human or animal;
putting said human or animal in an appropriate position in order to achieve the exposure of the rectosigmoidal transition; and
inserting at least one or more of the following into said natural body orifice with at least a portion extending past said rectosigmoidal transition:
(a) a trocar tube (2) comprising:
a distal portion (22) to be positioned at least in the region of or outside the body orifice; and
a proximal portion (21) to be positioned within the human or animal, wherein:
at least the proximal portion (21) comprises at least partially an essentially non-deformable curvature;
(b) a trocar comprising the trocar tube and a trocar head;
(c) an obturator (3) receivable into the trocar tube; and
(d) a rectoscope (5) having a port (55) into which the trocar is receivable.

36. The method for treatment and/or diagnosis according to claim 35, comprising the steps of:
(a) introducing the rectoscope (5) into the rectum;
(b) introducing an ultrasound probe (90) and a guide instrument (80) into the rectoscope;
(c) positioning a puncture needle with a guide wire at a desired site in the intestine by means of the guide instrument (80);
(d) removing the guide instrument and the ultrasound probe (90) and introducing the trocar comprising the obturator for piercing the intestine.

37. The method according to claim 36, further comprising:
leaving the guide wire in the rectoscope after step (c); and
steering the obturator along the guide wire to the desired site in the intestine.
